SEQUENCE LISTING (13 sequences)

SEQ ID NO: 1 - DNA, 24 bp, Artificial Sequence
  CSN4C2-A oligonucleotide of CSAD adaptor; Chemically synthesized
gccattaaggccaccatgccnnnn

SEQ ID NO: 2 - DNA, 41 bp, Artificial Sequence
  CSAD-A oligonucleotide of CSAD adaptor; Chemically synthesized
catggtggccttaatggccactacgaccgttcgggtggtac

SEQ ID NO: 3 - DNA, 24 bp, Artificial Sequence
  CSN4C2-A oligonucleotide of CSAD adaptor after annealing; Chemically synthesized
gccattaaggccaccatgccnnnn

SEQ ID NO: 4 - DNA, 41 bp, Artificial Sequence; identical to SEQ ID NO: 2
  CSAD-A oligonucleotide of CSAD adaptor after annealing; Chemically synthesized

SEQ ID NO: 5 - DNA, 21 bp, Artificial Sequence
  PSN4G2 oligonucleotide of PSAD adaptor; Chemically synthesized
nnnnggtgagtgactgaggcc

SEQ ID NO: 6 - DNA, 44 bp, Artificial Sequence
  PSAD oligonucleotide of PSAD adaptor; Chemically synthesized
cgaggagcgaccgactcgatggccgaggcggcctcagtcactca

SEQ ID NO: 7 - DNA, 21 bp, Artificial Sequence
  PSN4G2 oligonucleotide of PSAD adaptor after annealing; Chemically synthesized
nnnnggtgagtgactgaggcc

SEQ ID NO: 8 - DNA, 44 bp, Artificial Sequence; identical to SEQ ID NO: 6
  PSAD oligonucleotide of PSAD adaptor after annealing; Chemically synthesized

SEQ ID NO: 9 - DNA, 21 bp, Artificial Sequence
  Primer; Chemically synthesized
gtaccacccgaacggtcgtag

SEQ ID NO: 10 - DNA, 21 bp, Artificial Sequence
  Primer; Chemically synthesized
cgaggagcgaccgactcgatg

SEQ ID NO: 11 - DNA, 13 bp, Artificial Sequence
  Sfi I cleavage site; Chemically synthesized
ggccnnnnnggcc

SEQ ID NO: 12 - DNA, 13 bp, Artificial Sequence
  Sfi I cleavage site; Chemically synthesized
ggccnnnnnggcc

SEQ ID NO: 13 - DNA, 46 bp, Artificial Sequence
  pTZV-CMV-Sfi-SIN vector; Chemically synthesized
gaattggccattaaggcctgcaggatccggccgcctcggcctcgag

(19) United States
(12) Patent Application Publication
(10) Pub. No.: US 2008/0138863 A1
(43) Pub. Date: Jun. 12, 2008

(54) Methods for Cloning Nucleic Acids in a Desired Orientation

(75) Inventors: Paz Einat, Nes Zionna (IL); Dina Zevin-Sonkin, Rehovot (IL); Shlomit Gilad, Gedera (IL)

Correspondence Address: COOPER & DUNHAM, LLP, 1185 AVENUE OF THE AMERICAS, NEW YORK, NY, 10036, US

(73) Assignee: QUARK PHARMACUTICALS,INC., FREMONT, CALIFORNIA, CA (US)

(21) Appl. No.: 10/561,005

(22) PCT Filed: Jun. 15, 2004
(86) PCT No.: PCT/IL04/00515

§ 371 Date: Dec. 15, 2005

Related U.S. Application Data
(60) Provisional application No. 60/479,224, filed on Jun. 16, 2003.

(51) Int. Cl. C12N 15/64; C40B 40/06; C12N 9/00; C07H 21/04

(52) U.S. Cl. 435/91.41; 506/16; 435/183; 536/23.1

(57) ABSTRACT

The present invention provides highly accurate methods for cloning nucleic acids in a desired, pre-defined orientation, and additionally provides for a library of cloned oriented nucleic acids. The ability to control the orientation of a nucleic acid being cloned is highly important in various molecular biology applications.

### FIELD OF THE INVENTION

The present invention relates to the fields of molecular biology and genetic engineering. More specifically, the invention relates to the field of nucleic acid cloning.

### BACKGROUND OF THE INVENTION

A highly potent research tool designed in the past two decades in the field of molecular biology is that of nucleic acid cloning, which revolutionized the field and enabled many important discoveries.

Today, nucleic acid cloning is at the forefront of molecular biology, with countless variations existing, and the materials and methods involved are adapted for a large number of goal-oriented applications.

Cloning began to be widely employed as a research tool in the mid 1970s (see, for example, Paul J., Gene cloning in cell biology. Cell Biol Int Rep 1978 Jul;2(4):311-26; Vosberg HP., Molecular cloning of DNA. An introduction into techniques and problems, Hum Genet 1977 Dec 29;40(1):1-72 and the references therein). Since then, methodologies and applications have become diverse (see, for example, Sambrook et al., Molecular cloning: A laboratory manual, Cold Springs Harbor Laboratory, New-York (1989, 1992), and Ausubel et al., Current Protocols in Molecular Biology, John Wiley and Sons, Baltimore, Md. (1988, 1998).).

Cloning of nucleic acids in a desired orientation is relevant mainly to cloning of mRNA through their conversion to cDNA and has several important applications. First, in expression cloning, where the cDNA is to be expressed in vitro or in vivo, the orientation of the cDNA within the expression vector is critical. In some cases expression of the full protein is desired requiring a full-length cDNA to be in a sense orientation. In other cases small pieces of the full protein are to be expressed from small pieces of cDNA, again requiring the cDNA pieces to be in a sense orientation. In other cases the expression of antisense RNA is needed, either as full-length or in short pieces, requiring the cDNA to be in the antisense orientation in the expression vector. Another application is the establishment of EST type CDNA libraries. Such libraries are used for mass sequencing of cDNA fragments for the purpose of identifying many of the mRNAs expressed in cells or tissues. The ability to derive oriented fragments from “middle” parts of mRNAs and not only from the termini (“edges”) is important in enabling easier characterization of mRNAs.

Cloning of cDNA in a desired orientation, without using sequence knowledge, is primarily done using the polyA tail of the mRNA. Alternatively, the CAP site can be used for this purpose (Shibata Y, Carninci P, Watahiki A, Shiraki T, Konno H, Muramatsu M, Hayashizaki Y. Cloning full-length, cap-trapper-selected cDNAs by using the single-strand linker ligation method. Biotechniques 2001 June;30(6):1250-4).However, in both cases either a full-length cDNA is cloned or only cDNA fragments carrying these regions can be cloned in a pre-defined orientation.

While cloning is commonplace in the laboratory, it is not currently possible to clone nucleic acid fragments in a desired orientation (5′-3′, i.e. sense orientation; or 3′-5′, i.e. anti-sense orientation) unless they contain a specific region which can be recognized, such as a Poly-A region or a CAP region. Therefore, nucleic acid fragments that do not contain such recognizable regions cannot be cloned in a desired orientation; the orientation in which they will be cloned will be arbitrary, random and unknown. In some methodologies fragments which do not contain these recognizable regions may be lost and not cloned at all. In addition, a lot of time, money and effort may be spent in discovering the orientation of a nucleic acid isolated from a library. Researchers engaged in cloning as a routine practice must also frequently waste time and money on sequencing a clone or series of clones to make sure they select one in the correct orientation for continued work.

The orientation of the cDNA fragment is critical in applications in which expression libraries of cDNA fragments are used to interrogate cellular functions. The cDNA fragments are transcribed in the cells and inhibit the expression of matching endogenous genes (mRNAs). Inhibition can be caused when the cDNA fragment is in the antisense orientation, and the antisense mRNA transcribed from it inhibits the endogenous mRNA through base-pairing. Alternatively, sense oriented cDNA fragments can be translated into dominant negative peptides that inhibit the matching endogenous protein e.g. by competition or binding. In these applications genes involved in specific cellular functions are identified through the characterization of the cDNA fragments since in most cases only a match between the cDNA and the endogenous gene leads to inhibition. Some of these applications seek short dominant peptides that inhibit genes involved in specific cellular functions (Gudkov et al., PNAS USA 91; 3744-48 (1994)). In such cases, availability of expression libraries in which all cDNA fragments are in the sense orientation would be highly advantageous. Similarly, other applications seek to express antisense mRNAs that will inhibit genes and again, availability of expression libraries in which all fragments are in the antisense orientation is highly advantageous. Of special importance are cases in which the inhibition of specific genes causes cell death. Such cases exist in applications seeking to identify genes involved in cancer, which can serve as targets for the development of anticancer drugs. In such applications the inhibition of a gene by antisense mRNA or sense mRNA (through its translation into a dominant negative peptide) leads to cell death. Thus, the gene that is inhibited causes the cells harboring the expressed cDNA fragments to disappear from the cell culture. In such a “negative selection” process the cDNA fragments are identified based on their disappearance from the culture. However, if in the cDNA fragment library both sense and antisense fragments co-exist it may lead to inability to identify the disappearance of fragments. In most cases only one of the orientations leads to inhibition of endogenous gene and thus one of them will not be depleted from the culture and will mask the ability to detect the depletion of the other.

### SUMMARY OF THE INVENTION

The inventors of the instant application have discovered methods for cloning nucleic acids in a desired, pre-defined orientation; DNA libraries prepared according to these methods demonstrate that the accuracy of the method is close to 100%. In some of its embodiments, the present invention provides methods for cloning nucleic acids in a desired orientation.

In additional embodiments, the present invention provides kits for performing these methods. In another embodiment, the present invention provides a library of cloned oriented nucleic acids. In an additional embodiment, the present invention provides a method for digesting ssDNA using restriction enzymes that usually digest dsDNA.

### DETAILED DESCRIPTION OF THE INVENTION

Current cloning methods employ double stranded nucleic acids, and thus the directionality is lost as both sides have the same basic structure. The inventors of the instant application had the novel idea of using the directionality of single stranded nucleic acids (i.e., the 5′ phosphate on one terminus and 3′ hydroxyl [OH] on the other) for the differential ligation of oligonucleotides to the 3′ and 5′ termini of a single-stranded nucleic acid fragment. The attachment of an oligonucleotide to the 3′ terminus of a single-stranded nucleic acid can be achieved by the use of T4 RNA ligase (which ligates both single stranded RNA and single stranded DNA). Other options include the use of adaptors with a double stranded region and an over-hang for ligation with common DNA ligases like T4 DNA ligase. RNA or first strand single-stranded DNA can be digested into short fragments by the use of restriction enzymes capable of digesting single stranded nucleic acids; alternatively, single-stranded nucleic acids can be digested by restriction enzymes that digest double-stranded nucleic acids according to the process described below. The methods of the present invention can be adapted to any approach for the random cloning of fragments in an oriented fashion, including the preparation of oriented-fragment nucleic acid libraries. Optionally, one can make an oriented fragment library of one gene; this involves producing artificial RNA (aRNA) from the full-length gene using enzymes such as T7 and SP6 RNA polymerase, and using this as the starting material.

One embodiment of the present invention concerns a process or method of cloning a nucleic acid such as, inter alia, DNA in a desired orientation comprising:
(a) obtaining a single stranded fragment of the nucleic acid (such as DNA);
(b) ligating an oligonucleotide primer comprising at least one restriction enzyme recognition site to the 3′ terminus of the fragment;
(c) producing a double-stranded nucleic acid (such as DNA) using a primer complementary to the primer of step (b); and
(d) cloning the double-stranded nucleic acid into a desired vector.

This process is exemplified in FIG. 1, using the option of a restriction enzyme recognition site (step (b)) for the enzyme NotI and subsequently performing the cloning (step (d)) with NotI and EcoRV.

The nucleic acid fragment may be genomic DNA or cDNA; it is understood that this process can also be carried out on other nucleic acids such as RNA or synthetic polynucleotides/ oligonucleotides.

This process may be used to clone any single stranded nucleic acid with high efficiency, as will be exemplified below; the cloning process has the advantage of not being dependent on particular regions of the single stranded nucleic acid, the oligo used in step (b) is typically not a CAP based oligo.

The term “nucleic acid” as used herein encompasses “polynucleotide” and “oligonucleotide”, and refers to any molecule composed of DNA nucleotides, RNA nucleotides or a combination of both types, i.e. that comprises two or more of the bases guanidine, cytosine, thymidine, adenine, uracil or inosine, inter alia. A nucleic acid may include natural nucleotides, chemically modified nucleotides and synthetic nucleotides, or chemical analogs thereof. A polynucleotide generally has from about 75 to 10,000 nucleotides, optionally from about 100 to 3,500 nucleotides. An oligonucleotide, also termed “oligo” for short, refers generally to a chain of nucleotides extending from 2-75 nucleotides.

In addition, step (b) of the process may further comprise ligating a specific primer to the 5′ terminus of the fragment, which may comprise a restriction enzyme recognition site not present in the primer specific for the 3′ terminus of the fragment. Step (c) may further comprise using a primer complementary to this primer.

Further, the ligation of step (b) may be performed with T4 RNA ligase or other ligases as judged to be appropriate by one of skill in the art, and production of the second strand in step (c) (to form a double-stranded nucleic acid) may optionally be performed by polymerization with Klenow enzyme or other polymerases as judged to be appropriate by one of skill in the art.

Additionally, step (c) may be split into two stages of polymerization; in such a case, a first polymerization reaction is carried out, typically a short reaction of only several polymerization rounds; the products of this polymerization reaction are then purified and/or sorted according to size, for example on an agarose gel; a second polymerization reaction is subsequently carried out on the purified products of the first polymerization reaction.

Commercially available ligase enzymes include T4 DNA ligase, T4 RNA ligase, Taq DNA ligase, E. Coli DNA ligase, Pfu DNA ligase and Tth DNA ligase, inter alia. Commercially available polymerase enzymes include Taq DNA polymerase, Vent DNA polymerase, Deep vent DNA polymerase, Pfu DNA polymerase and Tth DNA polymerase, inter alia.

Methods of obtaining a single stranded nucleic acid are well known in the art and include physical shearing, enzymatic digestion (including digestion with S1 nuclease), de-purination and other methods. Any method that will produce nucleic acid fragments capable of being ligated to oligonucleotides or adaptors can optionally be used with the processes of the instant invention.

Digestion of a single stranded nucleic acid can be accomplished using restriction enzymes that digest single stranded nucleic acids, such as, inter alia: Hha I, HinPl I, MnI I, Hae III, BstN I, Dde I, Hga I, Hinf I and Taq I. In addition, digestion of single stranded nucleic acids can be accomplished using restriction enzymes that digest double stranded nucleic acids—according to the process described below—such as, inter alia: Aar I, Aas I, Aat II, Aau I, Acc I, Acc II, Acc III, Acc16 I, Acc36 I, Acc65 I, Acc113 I, AccBl I, AccB7 I, AccBS I, Aci I, Acl I, AclW I, Acs I, Acu I, Acv I, Acy I, Ade I, Afa I, Afe I, Afl I, Afl II, Afl III, Age I, Ahd I, Ahl I, Ale I, Alo I, Alu I, Alw I, Alw21 I, Alw26 I, Alw44 I, AlwN I, Ama87 I, Aoc I, Aor51H I, Apa I, ApaL I, Apo I, Asc I, Ase I, AsiA I, AsiS I, Asn I, Asp I, Asp700 I, Asp718 I, AspE I, AspH I, AspLE I, AspS9 I, Asu II, AsuC2 I, AsuHP I, AsuNH I, Ava I, Ava II, Avc I, Avi II, Avr II, Axy I, Bae I, Bal I, BamH I, Ban I, Ban II, Ban III, Bbe I, BbrP I, Bbs I, Bbu I, Bbv I, Bbv12 I, BbvC I, BceA I, Bcg I, BciV I, Bcl I, Bcn I, Bco I, Bcu I, Bfa I, Bfi I, Bfm I, Bfr I, BfrB I, Bfu I, BfuA I, BfuC I, Bgl I, Bgl II, Bln I, Blp I, Bme18 I, Bme1390 I, Bme1580 I, BmgB I, Bmr I, Bmt I, Bmy I, Box I, Bpi I, Bpl I, Bpm I, Bpu10 I, Bpu14 I, Bpu1102 I, BpuA I, BpuE I, Bsa I, Bsa29 I, BsaA I, BsaB I, BsaH I, BsaJ I, BsaM I, BsaO I, BsaW I, BsaX I, Bsc I, Bsc4 I, Bsel I, Bse3D I, Bse8 I, Bse21 I, Bse118 I, BseA I, BseB I, BseC I, BseD I, Bse3D I, BseG I, BseJ I, BseL I, BseM I, BseM II, BseN I, BseP I, BseR I, BseS I, BseX I, BseX3 I, BseY I, Bsg I, Bsh1236 I, Bsh1285 I, Bsh1365 I, BshF I, BshN I, BshT I, BsiB I, BsiE I, BsiHKA I, BsiHKC I, BsiM I, BsiS I, BsiW I, BsiY I, BsiZ I, BsI I, Bsm I, BsmA I, BsmB I, BsmF I, Bso31 I, BsoB I, Bsp13 I, Bsp19 I, Bsp68 I, Bsp106 I, Bsp119 I, Bsp120 I, Bsp143 I, Bsp143 II, Bsp1286 I, Bsp1407 I, Bsp1720 I, BspA2 I, BspC I, BspCN I, BspD I, BspE I, BspH I,BspL I, BspLU11 I, BspM I, BspP I, BspT I, BspT104 I, BspT107 I, BspTN I, BspX I, Bsr I, BsrB I, BsrBR I, BsrD I, BsrF I, BsrG I, BsrS I, BssA I, BssEC I, BssH I, BssH II, BssK I, BssNA I, BssS I, BssT1 I, Bst2B I, Bst2U I, Bst4C I, Bst6 I, Bst71 I, Bst98 I, Bst1107 I, BstAC I, BstAP I, BstB I, BstBA I, BstC8 I, BstDE I, BstDS I, BstE II, BstEN I, BstEN II, BstF5 I, BstFN I, BstH2 I, BstHH I, BstHP I, BstKT I, BstMA I, BstMC I, BstMW I, BstN I, BstNS I, BstO I, BstP I, BstPA I, BstSC I, BstSF I, BstSN I, BstU I, BstV1 I, BstV2 I, BstX I, BstY I, BstZ I, BstZ17 I, Bsu15 I, Bsu36 I, BsuR I, BsuTU I, Btg I, Btr I, Bts I, Cac8 I, Cai I, CciN I, Cel II, Cfo I, Cfr I, Cfr9 I, Cfr10 I, Cfr13 I, Cfr42 I, Cla I, Cpo I, Csp I, Csp6 I, Csp45 I, CspA I, CviJ I, CviR I, CviT I, Cvn I, Dde I, Dpn I, Dpn II, Dra I, Dra II, Dra III, Drd I, Dsa I, DseD I, Eae I, Eag I, Eam1104 I, Eam1105 I, Ear I, Eci I, Ec1136 II, EclHK I, EclX I, Eco24 I, Eco31 I, Eco32 I, Eco47 I, Eco47 III, Eco52 I, Eco57 I, Eco57M I, Eco72 I, Eco8 I, Eco88 I, Eco91 I, Eco105 I, Eco130 I, Eco147 I, EcoICR I, EcoN I, EcoO65 I, EcoO109 I, EcoR I, EcoR II, EcoR V, EcoT14 I, EcoT22 I, EcoT38 I, Ege I, Ehe I, Erh I, Esp3 I, Fal I, Fat I, Fau I, FauND I, Fba I, Fbl I, Fnu4H I, Fok I, FriO I, Fse I, Fsp I, Fsp4H I, FspA I, Fun I, Fun II, Gsu I, Hae II, Hae III, Hap II, Hga I, Hha I, Hin1 I, Hin4 I, Hin6 I, Hinc II, Hind II, Hind III, Hinf I, HinP1 I, Hpa I, Hpa II, Hph I, Hpy8 I, Hpy99 I, Hpy188 I, Hpy188 III, HpyCH4 III, HpyCH4 IV, HpyCH4 V, HpyF10 VI, Hsp92 I, Hsp92 II, HspA I, Ita I, Kas I, Kpn I, Kpn2, I Ksp I, Ksp22 I, Ksp632 I, KspA I, Kzo9 I, Lsp I, Lwe I, Mab I, Mae I, Mae II, Mae III, Mam I, Mbi I, Mbo I, Mbo II, Mfe I, Mfl I, Mhl I, Mls I, Mlu I, MluN I, Mly I, Mly113 I, Mme I, Mnl I, Mph1103 I, Mro I, MroN I, MroX I, Msc I, Mse I, Msl I, Msp I, Msp17 I, Msp20 I, MspAI I, MspC I, MspR9 I, Mss I, Mun I, Mva I, Mva1269 I, Mvn I, Mwo I, Nae I, Nar I, Nci I, Nco I, Nde I, Nde II, NgoA IV, NgoM IV, Nhe I, Nla III, Nla IV, NmuC I, Not I, Nru I, NruG I, Nsb I, Nsi I, Nsp I, Nsp III, Nsp V, Oli I, Pac I, Pae I, PaeR7 I, Pag I, Pal I, Pau I, Pce I, Pci I, Pct I, Pdi I, Pdm I, Pfl23 II, PflB I, PflF I, PflM I, PinA I, Ple I, Ple19 I, PmaC I, Pme I, Pml I, Ppi I, Pps I, Ppu10 I, PpuM I, PpuX I, PshA I, PshB I, Psi I, Psp5 II, Psp6 I, Psp124B I, Psp1406 I, PspA I, PspE I, PspG I, PspL I, PspN4 I, PspOM I, PspP I, PspPP I, Psr I, Pst I, Psu I, Psy I, Pvu I, Pvu II, Rca I, Rsa I, Rsr, Rsr2 I, Sac I, Sac II, Sal I, SanD I, Sap I, Sat I, Sau3A I, Sau96 I, Sbf I, Sca I, Sch I, ScrF I, Sda I, Sdu I, SexA I, SfaN I, Sfc I, Sfi I, Sfo , Sfr274 I, Sfr303 I, Sfu I, Sgf I, SgrA I, SgrB I, Sin I, Sla I, Sma I, Smi I, SmiM I, Sml I, Smu I, SnaB I, SpaH I, Spe I, Sph I, Srf I, Sse9 I, Sse8387 I, SseB I, Ssp I, SspB I, Sst I, Sst II, Stu I, Sty I, Sun I, Swa I, Taa I, Tai I, Taq I, Taq II, Tas I, Tat I, Tau I, Tel I, Tfi I, Tha I, Tli I, Tru1 I, Tru9 I, Tsc I, Tse I, Tsp45 I, Tsp509 I, TspDT I, TspE I, TspGW I, TspR I, Tth111 I, TthHB8 I, Van91 I, Vha464 I, Vne I, VpaK11B I, Vsp I, Xag I, Xap I, Xba I, Xce I, Xcm I, Xho I, Xho II, Xma I, Xma III, XmaC I, XnaJ I, Xmi I, Xmn I, Xsp I, Zho I, Zra I and Zsp2 I.

Ligation of an adaptor or oligonucleotide to the 3′ terminus of the single stranded nucleic acid fragment (such as ssDNA or ssRNA) is accomplished by modifying the oligonucleotide in such a way that it will ligate only to an OH (i.e., hydroxyl) tail. For this purpose, an oligonucleotide should have a phosphate on its 5′ terminus and should be blocked on its 3′ terminus. This blocking is necessary to ensure that the oligonucleotide will not ligate to the 5′ terminus of the single stranded nucleic acid. The general structure of an adaptor for ligation is presented in FIG. 4a.

Examples of the nucleotide structure of the overhang are described below. As can be seen, the 3′ terminus of the adaptor is blocked, so that the 3′ terminus cannot be used for ligation, and only the oligo strand intended to ligate to the 5′ end of the single stranded nucleic acid has the required OH (hydroxyl). A phosphate is added only to the 5′ terminus of the oligo that is intended to ligate to the 3′ terminus of the single stranded nucleic acid.

Methods of performing OH and phosphate modifications are well known in the art, and include, inter alia, synthesis with or without these groups, as desired, and, if necessary, use of polynucleotide kinase enzymes for example.

Protocols for performing ligation and amplification reactions are well known in the art and can be modified as desired by the skilled artisan.

An additional embodiment of the present invention provides a process or method of cloning a nucleic acid in a desired orientation comprising the steps of:
(a) obtaining a single stranded fragment of the nucleic acid;
(b) ligating a double stranded nucleic acid adaptor comprising at least one restriction enzyme recognition site to the termini of the fragment, wherein the adaptor ligated to the 5′ terminus and the adaptor ligated to the 3′ terminus differ in at least one restriction enzyme recognition site;
(c) amplifying the fragment by PCR with primers complementary to the adaptors of step (b), to obtain a double-stranded nucleic acid; and
(d) cloning the double-stranded nucleic acid into a desired vector.

This process may be used to clone any single stranded nucleic acid with high efficiency, as will be exemplified below; the cloning process has the advantage of not being dependent on particular regions of the single stranded nucleic acid, and the adaptor used in step (b) is typically not a Poly-T adaptor.

This process is exemplified in FIG. 2, using the option of a restriction enzyme recognition site (step (b)) for Not I in the adaptor that ligates to the 3′ terminus (“cap-side” adaptor) and a restriction enzyme recognition site for Asc I in the adaptor that ligates to the 5′ terminus (“polyA-side” adaptor) and subsequently performing the cloning (step (d)) with these two restriction enzymes.

The adaptor ligated to the 3′ terminus of the DNA fragment may have a 3′ nucleotide overhang, and the adaptor ligated to the 5′ terminus of the DNA fragment may have a 5′ nucleotide overhang; these overhangs optionally differ from each other in sequence.

The nucleic acid fragment may be genomic DNA or cDNA; it is understood that this process can also be carried out on other nucleic acids such as RNA or synthetic oligonucleotides.

After the single stranded nucleic acid fragment is obtained, it may optionally be cleaved into smaller fragments prior to the ligation of step (b). The adaptors used in the ligation of step (b) can in such a case further comprise the full or partial sequence of the restriction enzyme recognition site for the restriction enzyme used to cleave the fragment of step (a) into smaller fragments; this may facilitate adaptor binding during ligation (see Example 2).

Further, the ligation of step (b) may be performed with T4 DNA ligase. (This enzyme recognizes double-stranded DNA; hence, the adaptors are designed to form a double stranded region with the fragment).

Additionally, step (c) may be split into two stages of amplification; in such a case, a first amplification reaction is carried out, typically a short reaction of only several polymerization rounds; the products of this amplification reaction are then purified and/or sorted according to size, for example on an agarose gel; a second amplification reaction is subsequently carried out on the purified products of the first amplification reaction.

An additional embodiment of the present invention concerns an oriented nucleic acid library, optionally a cDNA library, prepared according to the methods of the present invention.

In an additional embodiment, the present invention provides a kit for performing the processes of the present invention comprising:
(a) A primer comprising at least one restriction enzyme recognition site that can ligate to the 3′ terminus of a nucleic acid; and
(b) T4 RNA ligase.

In another embodiment, the present invention provides a kit for performing the processes of the present invention comprising:
(a) A double stranded nucleic acid (optionally DNA) adaptor comprising at least one restriction enzyme recognition site and capable of ligating to the 5′ terminus of a single stranded nucleic acid;
(b) A double stranded nucleic acid (optionally DNA) adaptor comprising at least one restriction enzyme recognition site and capable of ligating to the 3′ terminus of a single stranded nucleic acid; and
(c) T4 DNA Ligase.

A cloning vector may optionally be included in these kits, comprising cloning sites compatible with the restriction enzyme recognition site in the 5′ and 3′ adaptors.

The present invention further provides a process or method of digesting a single stranded nucleic acid (such as DNA or RNA) with a restriction enzyme that digests double stranded nucleic acids, comprising annealing at least one oligonucleotide comprising a sequence recognized by a restriction enzyme that digests double stranded nucleic acids to the single stranded nucleic acid. Thus, the desired double-stranded restriction enzyme recognition site can be used to digest the single stranded nucleic acid; if necessary, the resultant fragments can subsequently be denatured and the oligos discarded. This method can help overcome the fact that only a few enzymes digest single stranded nucleic acids (and some of them have low efficiency).

More specifically, the oligonucleotides to be annealed to the single stranded nucleic acid are optionally non-palindromic, and/or may comprise non-complementary bases on either (or both) termini of the oligo, so as to prevent self annealing.

In an additional embodiment, the present invention provides adaptors for performing the processes of the invention; these adaptors may be included in any of the kits for performing the aspects of the invention, as described herein. One such adaptor is a double stranded nucleic acid adaptor comprising two oligonucleotides having a complementary region of 4 or more nucleotides and blocked 3′ termini, wherein one oligonucleotide has a 3′ overhang and lacks any phosphate on its 5′ terminus. Another such adaptor is a double stranded nucleic acid adaptor comprising two oligonucleotides having a complementary region of 4 or more nucleotides and lacking any phosphate on the 5′ termin, wherein one oligonucleotide has a 5′ overhang and a blocked 3′ terminus. Non-limiting examples of these adaptors are depicted in Example 2.

In both adaptors, one of the oligonucleotides may further comprise a single stranded region of 4 or more nucleotides; this region may be used for primer annealing in the later stages of the processes (such as amplification or polymerization).

The term “expression vector” as used herein refers to vectors that have the ability to incorporate and express heterologous nucleic acid fragments in a foreign cell. Many prokaryotic and eukaryotic expression vectors are known and/or commercially available. Selection of appropriate expression vectors is within the knowledge of those having skill in the art.

By “library” in the context of the above oriented fragment nucleic acid libraries, is meant a set of at least 5 nucleic acids that differ from each other. Libraries can include thousands, tens of thousands, hundreds of thousands and even millions of different elements.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

Throughout this application, various publications, including United States patents, are referenced by author and year and patents by number. The disclosures of these publications and patents and patent applications in their entireties are hereby incorporated by reference into this application in order to more fully describe the state of the art to which this invention pertains.

### BRIEF DESCRIPTION OF THE FIGURES

FIG. 1 is a flow chart describing the process of performing a method of the present invention using the exemplary ligase T4 RNA ligase and exemplary restriction enzymes.

FIG. 2 is a flow chart describing the process of performing a method of the present invention using the exemplary ligase T4 DNA ligase and exemplary restriction enzymes.

FIG. 3 depicts the vector (and its multiple cloning site) used to prepare the exemplary oriented fragment cDNA library described herein.

FIG. 4 depicts the structure of several adaptors which may be used with the methods of the present invention.

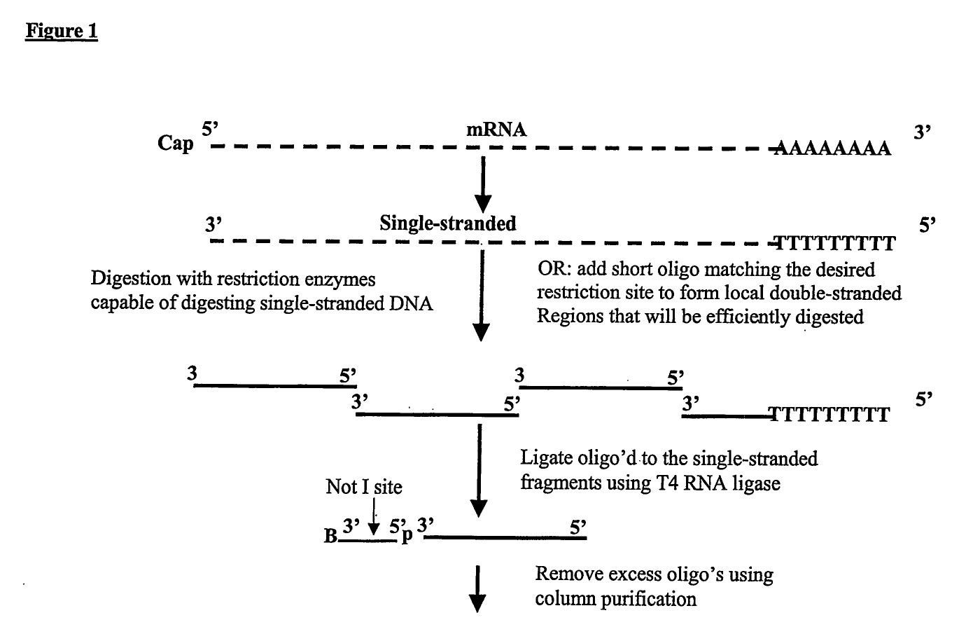

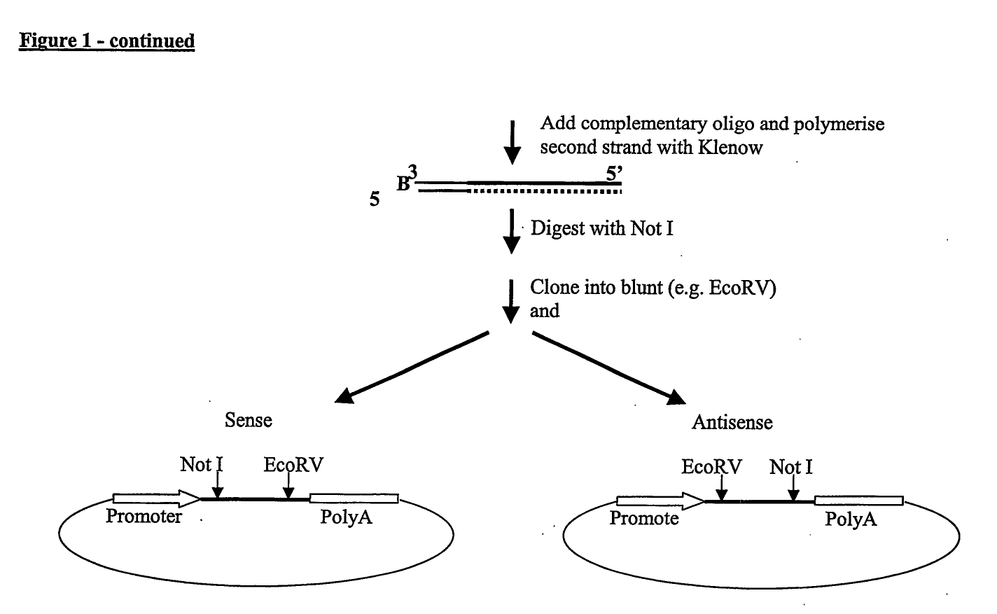

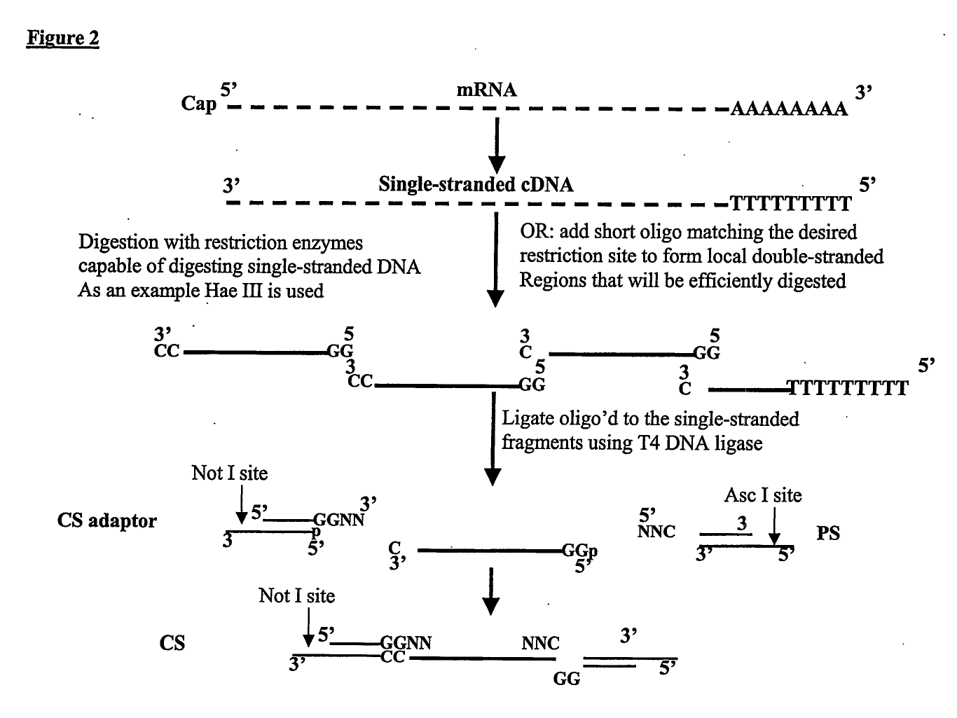

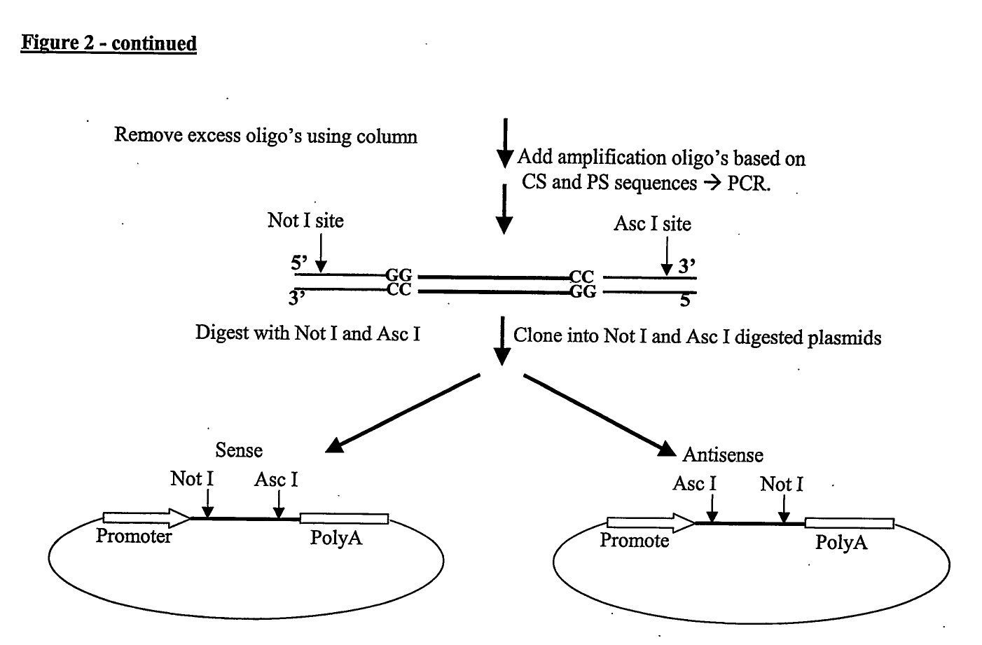

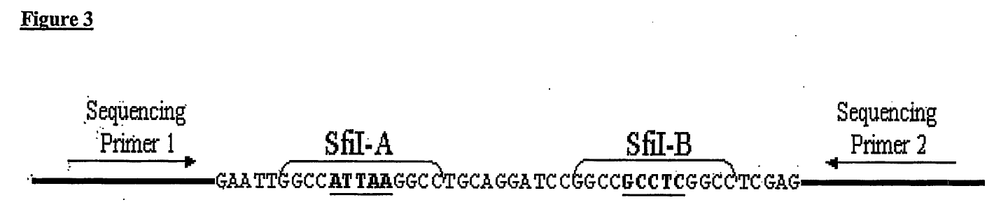

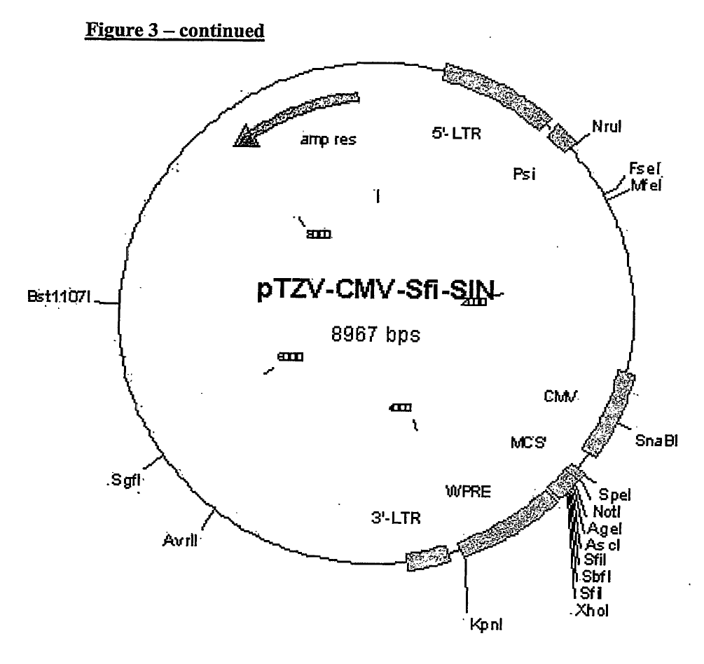

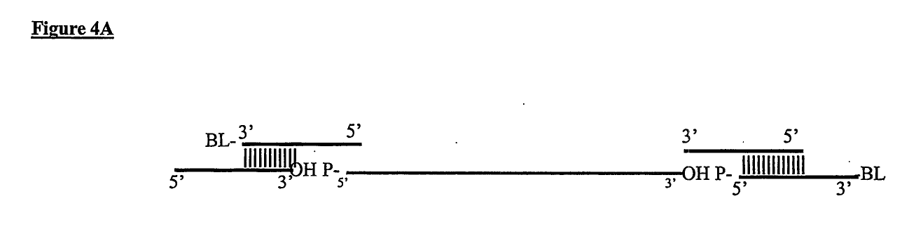

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

Standard molecular biology protocols known in the art not specifically described herein are generally followed essentially as in Sambrook et al., Molecular cloning: A laboratory manual, Cold Springs Harbor Laboratory, New-York (1989, 1992), and in Ausubel et al., Current Protocols in Molecular Biology, John Wiley and Sons, Baltimore, Md. (1988, 1998).

### Example 1

### Materials and protocols

I. Digestion of Single-Stranded cDNA

1. Produce single stranded cDNA from mRNA by reverse transcription.

2. Remove RNA and purify the DNA.

3. Digest single-stranded cDNA with HhaI, HinPI, HaeIII or MnlI (or any other enzyme capable of digesting single-stranded DNA). These enzymes are 4-bp cutters that cleave single-stranded DNA efficiently. [Another option is to add short oligonucleotides matching the digestion site and to use any chosen enzyme (e.g. use NAGCTN for AluI). This must be calibrated since such oligos produce double stranded DNA by themselves and can inhibit digestion of the cDNA (by competition) if used in excess.] In principle, any method for cleaving single-stranded DNA into short fragments, either enzymatic, chemical or mechanical - can be adapted to the procedure.

II. Ligation Using T4 RNA Ligase

1. Dephosphorylate single-stranded cDNA (necessary in the case where T4 RNA ligase is to be used, so as to prevent ligation of different cDNA fragments to each other and not to the oligonucleotide).

2. Purify.

3. Add LigOL and ligate to the single stranded fragments using T4 RNA ligase or T4 DNA ligase.

4. Purify to remove non-ligated LigOL.

5. Add EIOL and polymerize second strand with Klenow.

6. Digest double stranded fragments with NotI.

7. Clone into NotI—EcoRV (blunt) digested vector (any other enzymes can be used). If the desired orientation is antisense, the EcoRV site should be closer to the promoter and the NotI site closer to the PolyA signal region. If the desired orientation is sense, this order is reversed.

III. Ligation Using T4 DNA Ligase

The ligation step of the method of the present invention can also be accomplished using T4 DNA ligase.

1. Use two different adaptors for ligation to the two different ends of the single stranded cDNA fragment. The following design is for the use of HaeIII for digestion of the single-stranded cDNA. Any other enzyme or method of digestion can be easily applied—by making appropriate adaptors.

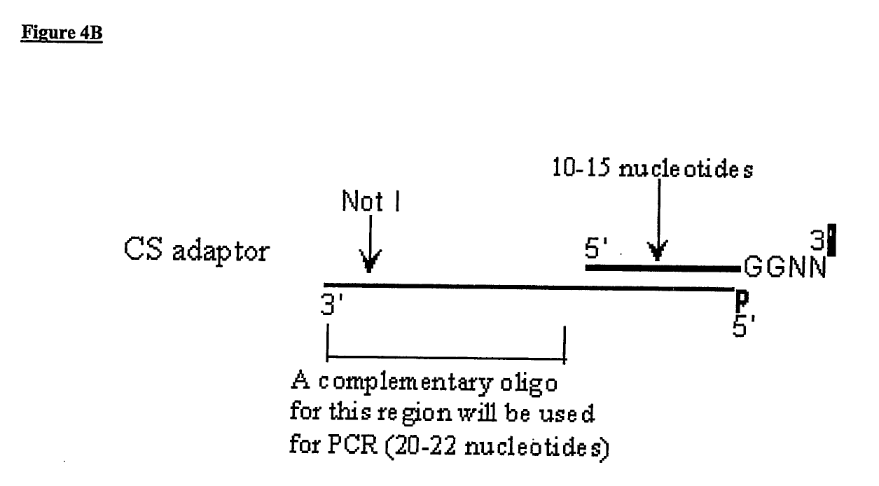

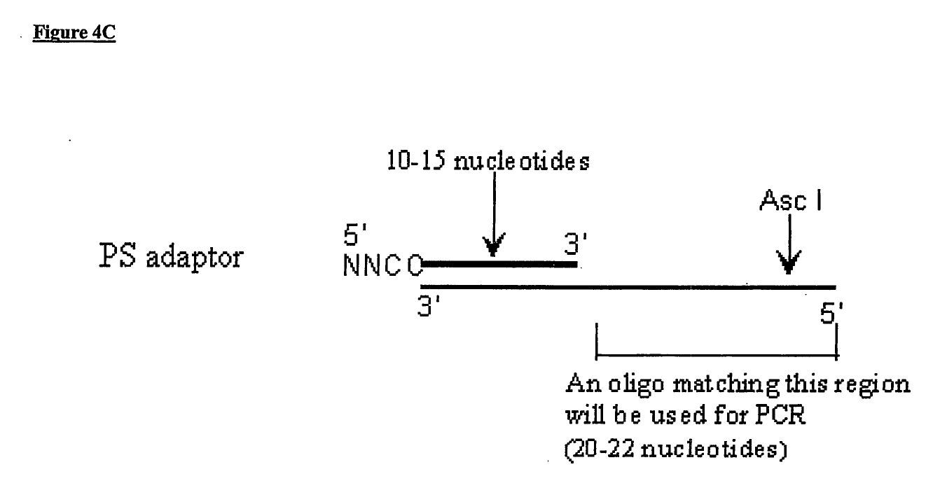

Cap-side (CS) adaptor will ligate only to the 3′ terminus of the single-stranded cDNA fragments. An optional structure for the CS adaptor is presented in FIG. 4b.

Notably, both 3′ termini of the adaptor are blocked, and the 5′ terminus on the short oligo does not have a phosphate.

The Poly-A side (PS) adaptor will ligate only to the 5′ terminus of the single-stranded cDNA fragments. An optional structure for the PS adaptor is presented in FIG. 4c.

Notably, both 5′ termini of the adaptor do not have phosphates, and the 3′ terminus on the short oligo is blocked.

2. Ligate the adaptors to the single-stranded cDNA fragments using T4 DNA ligase (or similar ligases). The adaptor design ensures that the CS adaptor will ligate only to the 3′ terminus of the single-stranded cDNA fragments and that the PS adaptor will ligate to the 5′ terminus of the single-stranded cDNA fragments.

3. Amplify the ligation products by PCR using the following two primers: one primer is complementary to the 3′ region of the CS adaptor (containing the NotI restriction site); the second primer matches the 5′ region of the PS adaptor (containing the AscI restriction site). It should be noted that the design does not permit the two adaptors to ligate to each other and thus prevents accumulation of a short, undesirable PCR product.

4. Purify the PCR products and digest with NotI and AscI. Any other enzyme combination that would allow directional cloning can be used.

5. Clone the digested products into a matching vector having the NotI and AscI restriction sites in its multiple-cloning region. The orientation of these cloning sites compared to the promoter and poly-A sequences in the expression vector will determine the orientation of the fragments.

### Example 2

### Preparation of an Anti-Sense cDNA Library Using the Processes of the Present Invention

A. Library Preparation.

An anti-sense library was prepared from mRNA derived from TGFb-treated rat NRK cells, according to the following protocol:

### 1. Reverse Transcription: PolyA+MRNA was Reverse Transcribed as Follows.

a. A total of 1 μg polyA+mRNA was annealed with 300 ng (150 pmol) of random hexamer (dN6) in a reaction volume of 17 μl. The mix was incubated for 2 minutes at 72° c and then placed on ice.

b. Reverse transcription reaction buffer (6 μl of 5x solution SuperScriptII buffer from Invitrogene) was added as well as DTT to a final concentration of 10 mM, NTP's to a final concentration of 0.5 mM, 40 units of RNAsin (Promega) and 200 units of SuperScriptII reverse transcriptase.

c. The reaction was incubated for 10 minutes at 25° c and then for 1 hour at 42° C. The reaction was then inactivated by incubation for 1.5 minutes at 70° c and then placed on ice.

### 2. Normalization of Single-Stranded CDNA.

a. To eliminate the mRNA, 2 units of RNAse H were added and the reaction incubated for 20 minutes at 37° C.

b. The single-stranded cDNA (sscDNA) was precipitated by adding 20 μg glycogen carrier and 100 μl of ethanol. Following centrifugation a pellet of sscDNA was obtained.

c. The Carninci normalization protocol (Carninci et al., Genome Research (2000),10:1617-1630) was followed. 3 μg biotinylated polyA+mRNA from the same source used for sscDNA synthesis was used for normalization. The sscDNA/biotinylated mRNA mix was hybridized till Rot=95 Mxsec. Non-hybridized cDNA was separated from hybridized on streptavidin magnetic beads (MPG Streptavidin beads from CPG inc.) and then precipitated and dissolved in 20 μl of H₂O.

3. HaeIII Digestion of sscDNA.

18 μl of the normalized sscDNA were incubated with 30 units of HaeIII (New England Biolabs) in buffer 2 (New England Biolabs) in a volume of 30 μl. The reaction was incubated over-night at 37° c and then inactivated for 15 minutes at 70° C.

### 4. Oligonucleotides and Adaptors.

a. Adaptors and oligonucleotides.

The following oligonucleotides constitute the adaptor (CSAD—cap side adaptor) that will ligate to the 3′ end (the CAP side with regard to the orientation of the mRNA) of the sscDNA.

CSN4C2-A
5′-GCCATTAAGGCCACCATGCCNNNN-3′
Block 24mer
CSAD-A
5′-p-CATGGTGGCCTTAATGGCCACTACGACCGTTCGGGTGGTAC-3′
Block 41mer

The structure of the CSAD adaptor after annealing is:

CSN4C2-A
5′GCCATTAAGGCCACCATGCCNNNN-3′
Block
CSAD-A
3′-CATGGTGGGCTTGCCAGCATCACCGGTAATTCCGGTGGTAC-P-5′

The following oligonucleotides constitute the adaptor (PSAD—poly A side adaptor) that will ligate to the 5′ end (the polyA side with regard to the orientation of the mRNA) of the sscDNA.

PSN4G2
5′-NNNNGGTGAGTGACTGAGGCC-3′
Block 21mer
PSAD
5′-CGAGGAGCGACCGACTCGATGGCCGAGGCGGCCTCAGTCACTCA-3′
44mer

The structure of the PSAD adaptor after annealing is:

PSN4G2
5′-NNNNGGTGAGTGACTGAGGCC-3′
Block
PSAD
3′-ACTCACTGACTCCGGCGGAGCCGGTAGCTCAGCCAGCGAGGAGC-5′

b. Oligonucleotide annealing to form the adaptors.

100 μm from each oligonucleotide of the pair that constitute an adaptor was mixed in 25 μl with an annealing buffer of 10 mM Tris-HCl, 7 mM MgCl₂, 100 mM NaCl.

The mix was placed in a 70° C. water bath previously switched off to permit cooling to room temperature.

### 5. Adaptor Ligation.

For ligation of adaptors to the ends of the sscDNA, the two adaptors were ligated in one reaction. It should be noted that the structure of the adaptors allow the CSAD to ligate only to the 3′ end of the sscDNA while the PSAD can ligate only to the 5′ end of the sscDNA. 2 μm from each adaptor were mixed with 13.5 μl of the HaeIII digested sscDNA from “3” above in the presence of ligation buffer and 800 units of T4 DNA ligase (New England Biolabs). Final reaction volume was 30 μl.

6. PCR Amplification of the Adaptor Ligated sscDNA.

The oligonucleotides used for PCR were:

| | | |
|---|---|---|
| CSPCR | 5′-GTACCACCCGAACGGTCGTAG-3′ | 21mer (for CSAD) |
| PSPCR | 5′-CGAGGAGCGACCGACTCGATG-3′ | 21mer (for PSAD) |

The PCR was performed in two steps so as to avoid formation of an adaptor dimer that could interfere with the amplification.

a. “Pre-PCR” for 3 cycles, without purification of the ligation reaction. A reaction volume of 100 μl included 15 μl of the ligation reaction, the PCR primers that match the adaptors: CSPCR and PSPCR oligonucleotides (final concentration of 0.4 mM), 0.2 mM dNTPs, ExTaq buffer and 3 units of ExTaq polymerase (TaKaRa). PCR cycling parameters were: 95° c 2′, 95° c 30″, 63° c 30″, 72° c 1 min, 3 cycles, 63° c 10′, hold 4° C.

b. The “pre-PCR” products were purified on GeneClean III and eluted in 30 μl 1 mM Tris pH 8.5.

c. Preparative PCR. Reaction volume of 50 μl included 27 μL of the purified “pre-PCR” products and the same reaction constituents as in “a”. PCR cycling parameters were: 95° C. 2′, 95° C. 30″, 63° C. 30″, 72° C. 1 min, 8 cycles, 63° C. 10′, hold 4° C. PCR products were precipitated with EtOH in the presence of 20 μg glycogen carrier. The pellet was dissolved in 20 μl DDW.

7. Agarose Gel Separation of Ligated cDNA.

The PCR products from the previous step were separated on 1.7% agarose gel. This is not an obligatory step but was done to further eliminate the free adaptors. The separated PCR products were divided into 3 fractions of size ranges of I. 450 to 900 bp, II. 300 to 450 bp, and III. 200 to 300 bp. DNA was extracted from gel with GeneClean Turbo and eluted in 30 μl Tris pH 8.5 10 mM. Yields were: fraction I. 35 ng, fraction II 100 ng, and fraction III 150 ng.

### 8. Amplification of Eluted PCR Products.

To obtain enough material for generating a representative high complexity library, the PCR products recovered from the agarose gel were further amplified. Roughly 13% of the recovered material was used in a PCR reaction with the same ingredients and volume as in 6a. Cycling parameters were as in 6a but 8 cycles were performed. After purification the yield was: fraction I 450 ng, fraction II 750 ng, and fraction III 2200 ng.

### 9. Description of Cloning Strategy Using Sfi I.

Ligated cDNA from each fraction was digested with Sfi I in preparation for ligation into the vector. Sfi I cleaves the following recognition sequence:

5′ . . . GGCCNNNN\hat{ }NGGCC . . . 3′
3′ . . . CCGGN\hat{ }NNNNCCGG . . . 5′

Thus, it generates a 3-basepair overhang of a sequence that is not part of the recognition sequence. This feature was utilized to generate two different cloning specificities in the two ends of the ligated cDNA. The CSAD generates an over-hang of:

3′AATTCCGG---

while the PSAD adaptor generates an over-hang of:

---GGCCCGGA-3′
---CCGGG-5′

Two matching Sfi I recognition sites were introduced into the cloning expression-vector pLNCX2 so that the PSAD side will be close to the promoter region in the cloning vector and the CSAD side will be close to the polyA region. This will cause the cDNA inserts to be in the ANTISENSE orientation in the cloning expression-vector.

Another advantage of using Sfi I is that its recognition sequence is composed of HaeIII cleavage recognition site (GGCC) and thus Sfi I will not cleave within the ligated cDNA (no internal SfiI sites are left and thus, no fragment is lost).

### 10. Digestion and Cloning.

The following amounts of adaptor-ligated cDNA were used for Sfi I digestion: 250 ng from fraction I, 425 ng from fraction II, and 1200 ng from fraction III. Following the digestion the products were purified by GeneClean Turbo kit and eluted in 30 μl of buffer.

### 11. Ligation and Transformation.

A dephosphorylated Sfi I digested pLNCX2 vector was used. The following table contains the detailed reaction conditions for each of the fractions.

TABLE 1

| | I | | II | | III | |
|---|---|---|---|---|---|---|
| PLNCX2/Sficut/CIP | 50 | ng | 50 | ng | 18 | ng |
| T4 DNA lig buf | 2 | ul | 2 | ul | 1 | ul |
| (NEB) ×10 | | | | | | |
| Fr I 450-900 bp | 36 | ng | | | | |
| Fr II. ~300-450 bp | | | 16.5 | ng | | |
| Fr III ~200-300 bp | | | | | 5 | ng |
| T4 DNA ligase (NEB) | 2 | ul | 2 | ul | 1 | ul |
| 400 u/ul | | | | | | |

| | | | |
|---|---|---|---|
| DDW | Up to 20 ul | Up to 20 ul | Up to 10 ul |

Ligation reactions were carried at room temperature for 4 hours.

Transformation was performed with the heat shock protocol according to Stratagene manual attached to the XL10-Gold Ultracompetent cells. Small amounts (1%, 0.1%) of each s transformation were plated for count and then large-scale plating was performed as detailed in Table 2 below. Colonies from the small scale transformation were taken for sequencing analysis of the library

TABLE 2

| | | |
|---|---|---|
| A | b | c |

| | | | | | | |
|---|---|---|---|---|---|---|
| XL10-Gold Ultra- | 230 | ul | 230 | ul | 100 | ul |
| competent cells | | | | | | |
| Spread on 15 cm | 4 | plates | 8 | plates | 2 | plates |
| plates | | | | | | |

| | | | |
|---|---|---|---|
| Colonies per plate | 31300 | 23000 | 32,000 |
| Total cfu | 125,000 | 140,000 | 64,000 |

### 12. Preparation of Library Plasmid DNA.

All the colonies obtained from the transformation above were collected and a ¼ of the bacteria were used for plasmid DNA preparation. The rest was stored as a glycerol stock. The plasmid DNA yield was 175 μg DNA. Thus, a library of a complexity of 330,000 clones was derived.

### B. Sequencing Analysis of Library.

The major importance of the sequencing analysis was to examine the efficiency of obtaining an oriented library. As described above, the library was designed to produce cDNA inserts in the antisense orientation. A primer from the promoter region was used for the sequencing reactions. Thus, the orientation of the sequences in the plasmid, relative to the direction of the “open-reading-frame” (ORF) of the mRNA from which the insert was derived, can be easily determined. Sequences were obtained from a total of 480 plasmids from the library. Only sequences that matched mRNAs of known orientation (i.e., genes contained in the RefSeq database possessing a clear open-reading-frame) were considered for assessment of the orientation. A total of 282 inserts matched mRNAs of known orientation. The annotation showed that 268 inserts were in the antisense orientation and 14 inserts in the sense orientation. Thus 95% of the library is in the expected orientation. It must be noted that there are increasing reports regarding the wide existence of natural antisense RNAs in cells (e.g. Yelin et al., Nature Biotech (2003) 21: 379 Widespread accurance of antisense transcription in the human genome). It is possible that at least some of the 14 inserts found in the “wrong” orientation could be derived from such natural antisense RNAs. Thus, the methods of the present invention allow for the preparation of an oriented library at very high efficiency.

The analysis of matches between HaeIII recognition sites and the ends of the inserts show that the digestion of the single-stranded cDNA with HaeIII was very efficient and in all cases in which the insert had a match to a rat mRNA in the database the ends were as expected. Only 10 inserts had an internal HaeIII fragment showing 3.5% partial digest. This can easily be avoided by increasing enzyme concentration or incubation time during the digestion of single-stranded cDNA.

### Example 3

### Preparation of Polynucleotides

The polynucleotides of the subject invention can be constructed by using a commercially available DNA synthesizing machine; overlapping pairs of chemically synthesized fragments of the desired polynucleotide can be ligated using methods well known in the art (e.g., see U.S. Pat. No. 6,121,426).

Another means of isolating a polynucleotide is to obtain a natural or artificially designed DNA fragment based on that sequence. This DNA fragment is labeled by means of suitable labeling systems which are well known to those of skill in the art; see, e.g., Davis et al. (1986). The fragment is then used as a probe to screen a lambda phage cDNA library or a plasmid cDNA library using methods well known in the art; see, generally, Sambrook et al., Molecular Cloning: A Laboratory Manual, Cold Spring Harbor Laboratory Press, New York (1989), in Ausubel et al., Current Protocols in Molecular Biology, John Wiley and Sons, Baltimore, Maryland (1989).

Colonies can be identified which contain clones related to the cDNA probe and these clones can be purified by known methods. The ends of the newly purified clones are then sequenced to identify full-length sequences. Complete sequencing of full-length clones is performed by enzymatic digestion or primer walking. A similar screening and clone selection approach can be applied to clones from a genomic DNA library.

1. A process of cloning a nucleic acid in a desired orientation comprising the steps of:
(a) obtaining a single stranded fragment of the nucleic acid;
(b) ligating an oligonucleotide primer comprising at least one restriction enzyme recognition site to the 3′ terminus of the fragment;
(c) producing a double-stranded nucleic acid using a primer complementary to the primer of step (b); and
(d) cloning the double-stranded nucleic acid into a desired vector.

2. The process of claim 1 wherein the nucleic acid is genomic DNA.

3. The process of claim 1 wherein the nucleic acid is cDNA.

4. The process of claim 1 wherein the nucleic acid is aRNA.

5. The process of claim 1 wherein step (b) further comprises ligating a specific primer to the 5′ terminus of the fragment.

6. The process of claim 1 wherein step (c) further comprises using a primer complementary to the primer of claim 5.

7. The process of claim 5 wherein the primer comprises a restriction enzyme recognition site not present in the primer specific for the 3′ terminus of the fragment.

8. The process of claim 1 wherein the ligation of step (b) is performed with T4 RNA ligase.

9. The process of claim 1 wherein step (c) is performed by polymerization with Klenow enzyme.

10. A process of cloning a nucleic acid in a desired orientation comprising the steps of:
(a) obtaining a single stranded fragment of the nucleic acid;
(b) ligating a double stranded adaptor comprising at least one restriction enzyme recognition site to each end of the fragment, wherein the adaptor ligated to the 5′ terminus and the adaptor ligated to the 3′ terminus differ in at least one restriction enzyme recognition site;
(c) amplifying the fragment by PCR with a primer complementary to a portion of the adaptor of step (b) ligated to the 5′ terminus and a primer complementary to a portion of the adaptor of step (b) ligated to the 3′ terminus, to obtain a double-stranded nucleic acid; and
(d) cloning the double-stranded nucleic acid into a desired vector.

11. The process of claim 10 further wherein the adaptor ligated to the 3′ terminus of the fragment in step (b) has a 5′ nucleotide overhang.

12. The process of claim 11 further wherein the adaptor ligated to the 5′ terminus of the fragment in step (b) has a 3′ nucleotide overhang.

13. The process of claim 10 wherein the adaptors ligated to both ends of the fragment have nucleotide overhangs that differ from each other in sequence.

14. The process of claim 10 wherein the nucleic acid is genomic DNA.

15. The process of claim 10 wherein the nucleic acid is cDNA.

16. The process of claim 10 wherein the nucleic acid is aRNA.

17. The process of claim 10 wherein the ligation of step (b) is performed with T4 DNA ligase.

18. The process of claim 10 further comprising digesting the fragment of step (a) into smaller fragments using a restriction enzyme.

19. The process of claim 18 wherein the adaptors used in step (b) further comprise the full or partial sequence of the restriction enzyme recognition site for the restriction enzyme used to digest the fragment of step (a) into smaller fragments.

20. A DNA library prepared according to the process of claim 1.

21-27. (canceled)

28. A DNA library prepared according to the process of claim 10.
